(19) United States
(12) Patent Application Publication
(10) Pub. No.: US 2026/0250766 A1
(43) Pub. Date: Aug. 27, 2026

(54) USE OF EXTRACELLULAR VESICLES AS BIOMARKERS FOR AGE-RELATED MACULAR DEGENERATION

(71) Applicants: The Regents of the University of Colorado, a body Corporate, Denver, CO (US); Research Foundation The City University of New York, New York, NY (US)

(72) Inventors: Stephen M. Redenti, Sea Cliff, NY (US); Miguel Flores-Bellver, Centennial, CO (US)

(21) Appl. No.: 19/162,025

(22) PCT Filed: Mar. 4, 2024
(86) PCT No.: PCT/US2024/018405

§ 371 Date: Sep. 3, 2025

Related U.S. Application Data
(60) Provisional application No. 63/449,893, filed on Mar. 3, 2023.

(51) Int. Cl. C12Q 1/6883; G01N 33/68

(52) U.S. Cl. CPC C12Q 1/6883; G01N 33/6893; C12Q 2600/158; C12Q 2600/178; G01N 2800/164; G01N 2800/52

(57) ABSTRACT

Methods of detecting various types of AMD and the likelihood of its progression to a more severe or advanced form of AMD involve detecting the presence or expression of one or more newly discovered AMD biomarkers contained within extracellular vesicles obtained from a subject. Biological samples containing the extracellular vesicles include fluid samples derived from plasma, urine, tears, saliva, or serum. Accurately diagnosing a particular form of AMD allows for the selection of treatment approaches not available or likely to be determined without the diagnosis.

### CROSS-REFERENCE TO RELATED APPLICATIONS

This application claims priority to U.S. Provisional Patent Application No. 63/449,893, titled “USE OF EXOSOME AS BIOMARKERS FOR AGE-RELATED MACULAR DEGENERATION” and filed on Mar. 3, 2023, the entire contents of which are incorporated by reference herein.

### TECHNICAL FIELD

The disclosed systems, methods, and devices are directed to the identification, detection, and use of biomarkers for age-related macular degeneration.

### BACKGROUND

Age-related macular degeneration (“AMD”) is the leading cause of irreversible visual impairment in the elderly population. AMD is a devastating disease that affects 11 million Americans, and this number is expected to double in the next 20 years. The number of people living with macular degeneration is expected to reach 288 million people worldwide by 2040. The estimated annual economic burden of vision loss and eye diseases in the United States is $140 billion, of which AMD is a major proportion. In the early stages of AMD, drusen accumulate between the retinal pigment epithelium and Bruchs membrane, compromising cell function and leading to oxidative stress, inflammation, and a maladaptive immune response. Notably, large drusen are associated with an increased risk of developing severe AMD, namely, neovascular AMD and geographic atrophy AMD. Predicting the development of one or more forms of AMD, including its initial diagnosis, has been elusive, as have corresponding treatments.

Accordingly, new techniques for accurately diagnosing and monitoring AMD, predicting its likelihood of progressing into a more severe or advanced form, and treating the condition are needed.

### SUMMARY

This disclosure provides systemic assays designed to identify and monitor specific stages of AMD in subjects afflicted with the condition. Embodiments involve performing a fluid biopsy on a subject, isolating the extracellular vesicles (“EVs”), e.g., exosomes, contained therein, and detecting one or more EV-derived biomarkers of AMD present within the sample. In various examples, the newly discovered biomarkers may include proteins, miRNAs, and/or metabolites detected within purified EVs present within a biofluid, such as plasma. The detection of one or more biomarker signatures disclosed herein can be used to diagnose a subject with one or more AMD subtypes, predict the progression of the disease, and design personalized treatment regimens aimed at inhibiting that progression.

In accordance with embodiments disclosed herein, a method of age-related macular degeneration assessment involves obtaining a biological sample from a subject, capturing at least one biomarker indicative of one or more subtypes of age-related macular degeneration from the biological sample, determining a presence or a level of the at least one biomarker in the biological sample, and comparing the presence or level of the at least one biomarker to a reference presence or level to determine whether the subject has age-related macular degeneration.

In some embodiments, age-related macular degeneration is intermediate age-related macular degeneration. In accordance with such embodiments, the at least one biomarker can include one or more of CALL5, TPM4, CDSN, K2C1B, SSC5D, IMP4, FCN3, miR-181c-5p, miR-1307-5p, miR-203a-3p, and L-cystine.

In some embodiments, age-related macular degeneration is neovascular age-related macular degeneration. In accordance with such embodiments, the at least one biomarker can include one or more of SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and 2-oxoglutaramate.

In some embodiments, age-related macular degeneration is geographic atrophy. In accordance with such embodiments, the at least one biomarker can include one or more of SSC5D, YWHAZ, APOC4, ANT3, TTR, VTN, miR-494-3p, miR-10399-5p, miR-625-5p, and phosphoenolpyruvate.

In some embodiments, the at least one biomarker can include one or more of miR-107, miR-181a-5p, miR-148a-3p, and miR-769-5p.

In some embodiments, the biological sample is a biofluid, such as plasma. In some embodiments, capturing at least one biomarker from a biological sample involves isolating extracellular vesicles present within the biological sample. In some embodiments, a method of age-related macular degeneration assessment further involves identifying a cargo contained within the extracellular vesicles, the cargo comprising protein, miRNA, and metabolites. In some embodiments, obtaining a biological sample and measuring the level of the at least one biomarker occurs at point of care. In some embodiments, a method of age-related macular degeneration assessment further involves determining whether administering a drug formulated to treat age-related macular degeneration will be effective. In some embodiments, a method of age-related macular degeneration assessment further involves implementing a treatment based on whether the subject is afflicted with a particular subtype of age-related macular degeneration.

In accordance with embodiments disclosed herein, a system for identifying a subject afflicted with age-related macular degeneration includes a device for receiving a biological sample from a subject suspected of having one or more subtypes of age-related macular degeneration, a device for identifying the presence of least one biomarker in the biological sample obtained from the subject, and a device for measuring the level of the at least one biomarker in the biological sample.

In some embodiments, the biological sample is a fluid sample, which may include one or more of plasma, urine, tears, saliva, aqueous humor, vitreous, or serum. In some embodiments, one or more subtypes of age-related macular degeneration include intermediate age-related macular degeneration, neovascular age-related macular degeneration, and geographic atrophy. In some embodiments, at least one biomarker includes one or more of CALL5, TPM4, CDSN, K2C1B, SSC5D, IMP4, FCN3, miR-181c-5p, miR-1307-5p, miR-203a-3p, L-cystine, SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, 2-oxoglutaramate, YWHAZ, APOC4, ANT3, TTR, VTN, miR-494-3p, miR-10399-5p, and miR-625-5p.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description, and no limitation as to the scope of the present disclosure is intended by either the inclusion or non-inclusion of elements, components, etc. in this Summary. Other features and advantages of the disclosed embodiments will be apparent from the following Detailed Description and claims.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the Detailed Description of certain embodiments presented herein.

FIG. 1A is a Venn diagram showing total spectral protein counts found in healthy individuals versus individuals having age-related macular degeneration (“AMD”), in accordance with embodiments disclosed herein.

FIG. 1B is a Venn diagram showing proteins overlapping and differing between healthy individuals relative to subjects with intermediate AMD (“iAMD”), neovascular AMD (“NV”), and geographic atrophy (“GA”), in accordance with embodiments disclosed herein.

FIG. 1C is a Venn diagram showing total detectable miRNAs found in healthy individuals versus all AMD conditions, in accordance with embodiments disclosed herein.

FIG. 1D is a Venn diagram showing miRNAs overlapping and differing between healthy individuals compared to subjects with iAMD, NV, and GA, in accordance with embodiments disclosed herein.

FIG. 2A is a volcano plot showing proteins expressed highly in healthy individuals versus subjects with iAMD, in accordance with embodiments disclosed herein.

FIG. 2B is a diagram showing seven proteins expressed significantly higher in healthy individuals versus iAMD subjects, in accordance with embodiments disclosed herein.

FIG. 2C is a diagram showing 34 proteins expressed significantly higher in iAMD subjects versus healthy individuals, in accordance with embodiments disclosed herein.

FIG. 3A is a volcano plot showing the proteins expressed highly in healthy individuals versus subjects with GA, in accordance with embodiments disclosed herein.

FIG. 3B is a diagram showing 15 proteins expressed significantly higher in healthy individuals compared to GA subjects, in accordance with embodiments disclosed herein.

FIG. 3C is a diagram showing six proteins expressed significantly higher in GA subjects compared to healthy individuals, in accordance with embodiments disclosed herein.

FIG. 4 includes a volcano plot showing proteins highly expressed in healthy individuals versus subjects with NV, along with a diagram (left) showing 12 proteins expressed significantly higher in healthy individuals relative to NV subjects, in accordance with embodiments disclosed herein.

FIG. 5 includes a volcano plot (center) highlighting the miRNAs highly expressed in healthy individuals versus subjects with iAMD, along with a diagram (right) that shows seven miRNAs expressed significantly higher in iAMD subjects compared to healthy individuals (CT), and a diagram (left) showing the sole miRNA expressed significantly higher in healthy individuals versus iAMD subjects, in accordance with embodiments disclosed herein.

FIG. 6A is a volcano plot highlighting the miRNAs expressed highly in healthy individuals versus subjects with GA, in accordance with embodiments disclosed herein.

FIG. 6B is a diagram showing nine miRNAs expressed significantly higher in healthy individuals compared to GA subjects, in accordance with embodiments disclosed herein.

FIG. 6C is a diagram showing 12 miRNAs expressed significantly higher in GA subjects compared to healthy individuals, in accordance with embodiments disclosed herein.

FIG. 7 includes a volcano plot (center) highlighting the miRNAs expressed highly in healthy individuals versus subjects with NV, along with a diagram (right) showing four miRNAs expressed significantly higher in NV subjects compared to healthy individuals, in accordance with embodiments disclosed herein.

FIG. 8A is a volcano plot highlighting the miRNAs expressed highly in GA subjects versus subjects with iAMD, in accordance with embodiments disclosed herein.

FIG. 8B is a diagram showing two miRNAs expressed significantly higher in GA subjects versus iAMD subjects, in accordance with embodiments disclosed herein.

FIG. 8C is a diagram showing five miRNAs expressed significantly higher in iAMD subjects compared to GA subjects, in accordance with embodiments disclosed herein.

FIG. 9 includes a volcano plot highlighting the miRNAs expressed highly in GA subjects versus subjects with NV, along with a diagram showing eight miRNAs expressed significantly higher in GA subjects versus NV subjects, in accordance with embodiments disclosed herein.

FIG. 10 is a Venn diagram showing the separate protein cargos within plasma-derived EVs collected from subjects with iAMD, NV, and GA, in accordance with embodiments disclosed herein.

### DETAILED DESCRIPTION

Disclosed herein are assays, systems, methods, and devices useful in diagnosing subjects with AMD earlier than previously achieved, determining the likely path of AMD progression, monitoring the condition over time, and administering treatments that promote improved outcomes in subjects afflicted with specific subtypes of AMD, including subjects likely to develop a more severe or advanced form of the condition. Embodiments include systemic assays that involve obtaining EVs from biological fluid samples, non-limiting examples of which can include plasma, urine, tears, saliva, aqueous humor, vitreous, or serum, and detecting previously undiscovered, stage-specific AMD biomarkers within the EV cargo. Disclosed biomarkers include proteins, miRNAs, and metabolites, different combinations of which, or “signatures,” uniquely corresponding to specific forms of AMD, including intermediate AMD, neovascular or “wet” AMD, and geographic atrophy or “dry” AMD. Obtaining bodily fluid samples from subjects and subsequently detecting the EV-derived biomarkers in the manner disclosed herein may lead to early diagnosis of AMD, which may inform a personalized treatment approach implemented in accordance with embodiments disclosed herein. Extracellular vesicles may thus provide surrogate markers for each AMD subtype obtainable via non-invasive means.

### Definitions

Unless defined otherwise below, all technical and scientific terms used herein have the same meaning as commonly understood by one of ordinary skill in the art to which this disclosure belongs.

### ABBREVIATIONS

MD=macular degeneration
AMD=age-related macular degeneration
iAMD=intermediate AMD
NV=neovascular or “wet” AMD
GA=geographic atrophy or “dry” AMD
EV=extracellular vesicle
CALL5=calmodulin-like protein 5
TPM4=tropomyosin alpha-4
CDSN=corneodesmosin
K2C1B may also be referred to as KRT77=keratin 77
SSC5D=scavenger receptor cysteine rich family member with 5 domains
IMP4=U3 small nucleolar ribonucleoprotein protein
FCN3=ficolin-3
SLC4A1=solute carrier family 4 member 1
LV746=immunoglobulin lambda variable 7-46
KV621=immunoglobulin kappa variable 6-21
YWHAZ=tyrosine 3-monooxygenase/tryptophan 5-monooxygenase activation protein
APOC4=apolipoprotein C-IV
ANT3=adenine nucleotide translocase 3
TTR=transthyretin
VTN=vitronectin
miRNA=microRNA
FAF=fundus autofluorescence
NIR=near infrared
OCT=optical coherence tomography

A “biomarker” is a characteristic that is objectively measured and evaluated as an indicator of normal biological processes, pathogenic processes, or pharmacological responses to a therapeutic intervention. Biomarkers may be of several types: indicative, predictive, prognostic, or pharmacodynamic (“PD”). Indicative biomarkers indicate whether a subject is afflicted with a specific condition, such as AMD. Predictive biomarkers predict which patients are likely to respond to or benefit from a particular therapy. Prognostic biomarkers predict the likely course of the patient's condition and may guide treatment. Pharmacodynamic biomarkers confirm drug activity and enable optimization of dose and administration schedule.

Various biomarkers useful in determining whether a subject has one or more forms of AMD, including whether the subject is susceptible to, or will, develop a more severe or advanced form of AMD, are disclosed herein. Embodiments include biomarkers selected from one or more proteins, microRNAs, metabolites, combinations thereof, and/or fragments thereof. Examples of protein biomarkers disclosed herein include one or more of CALL5, TPM4, CDSN, K2C1B, SSC5D, IMP4, FCN3, SLC4A1, LV746, KV621, YWHAZ, APOC4, ANT3, TTR, and/or VTN. Examples of microRNA biomarkers disclosed herein include one or more of miR-181c-5p, miR-1307-5p, miR-203a-3p, miR-107, miR-181a-5p, miR-148a-3p, miR-769-5p, miR-656-3p, miR-204-5p, miR-382-3p, miR-494-3p, miR-10399-5p, and/or miR-625-5p. Examples of metabolite biomarkers disclosed herein include one or more of L-cystine, indol pyruvate, octenoyl-l-carnitine, tetrahydrofolate, phosphoenolpyruvate, eicosapentaenoic acid, dimethylglycine, serotonin, L-citrulline, L-histidine, and/or 2-oxoglutaramate.

Groups or panels of one or more biomarkers may be indicative of the presence of AMD in a subject, including whether the subject is susceptible to, or will, develop a more severe or advanced form of AMD. For instance, one non-limiting example of a group or panel of biomarkers indicative of iAMD may include one or more, including all, of CALL5, TPM4, CDSN, K2C1B, SSC5D, IMP4, FCN3, miR-181c-5p, miR-1307-5p, miR-203a-3p, and/or L-cystine. One non-limiting example of a group or panel of biomarkers indicative of AMD may include one or more, including all, of miR-107, miR-181a-5p, miR-148a-3p, and/or miR-769-5p. One non-limiting example of a group or panel of biomarkers indicative of NV may include one or more, including all, of SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and/or 2-oxoglutaramate. One non-limiting example of a group or panel of biomarkers indicative of GA may include one or more, including all, of SSC5D, YWHAZ, APOC4, ANT3, TTR, VTN, miR-494-3p, miR-10399-5p, miR-625-5p, and/or phosphoenolpyruvate.

As used herein, the term “reference level” may refer to an established level and/or range of a biomarker (e.g., CALL5) in subjects not afflicted with AMD or a particular subtype or stage of AMD (e.g., iAMD).

In some examples, biomarker “detection” may refer to detecting the presence of one or more biomarkers within an EV-derived sample. Additionally or alternatively, biomarker “detection” may refer to determining one or more of an expression level, relative expression level, amount, and/or relative amount of one or more biomarkers within an EV-derived sample, and optionally comparing one or more of such levels and/or amounts to one or more corresponding reference levels. In some examples, biomarker “detection” may refer to detecting, measuring, or otherwise identifying one or more of the presence, expression level, relative expression level, amount, and/or relative amount of one or more biomarkers within an EV-derived sample.

The term “biological sample” or “sample” refers to a specimen obtained from a subject for use in the present methods, and includes various fluid samples, such as plasma, urine, tears, serum, aqueous humor, vitreous, and/or saliva.

As used herein, “extracellular vesicles” or “EVs” may include one or more subtypes, such as exosomes. The terms “EVs” and “exosomes” may be referred to interchangeably, despite exosomes constituting a subset of EVs. Embodiments of the systems and methods disclosed herein may encompass the use of EVs, broadly, or exosomes, more specifically.

Various methods of analyzing a biological sample for the presence, expression level, relative expression level, amount, and/or relative amount of one or more biomarkers are disclosed. In some embodiments, the method of analysis may be one or more of RNA sequencing, Western blotting, ECL, ELISA, Ella, Surface Plasmon Resonance, molecular array, SIMOA, or PCR. In embodiments, the analysis may include capturing the biomarker with an antigen binding protein or monoclonal antibody specific to the biomarker, wherein the capturing may be permanent or semipermanent. In some embodiments, the antigen binding protein or antibody may be immobilized on a substrate. The analysis may, in some embodiments, result in a value that reflects the concentration of the biomarker in the biological sample. In other embodiments, the level of the biomarker may be relative to other biomarkers, for example a reference biomarker. In some embodiments, the biological sample is a blood plasma sample, including one or more extracellular vesicles, e.g., exosomes, contained therein.

Confirmation of AMD in a subject, which may include a specific subtype or stage of AMD and a likelihood of the subject developing a severe or more advanced form of AMD, may be associated with an elevated level of one or more of the disclosed biomarkers, or biomarker signatures as a whole, relative to at least one reference level or threshold. In some embodiments, the reference level may be determined from one or more studies of healthy controls, which may include subjects not having AMD and/or one or more control subjects afflicted with a particular type of AMD, e.g., NV or GA, relative to a type of interest, e.g., iAMD.

As used herein, the terms “treat,” “treating,” “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition or disorder.

The terms “treat”, “treating” and “treatment” may further refer to eliminating, reducing, suppressing, or ameliorating, either temporarily or permanently, either partially or completely, a clinical symptom, manifestation or progression of an event, disease or condition associated with the diseases described herein, such as vision loss associated with AMD. As is recognized in the pertinent field, methods and drugs employed as therapies may reduce the severity of a given disease state, but need not abolish every manifestation of the disease to be regarded as useful. Similarly, a prophylactically administered treatment need not be completely effective in preventing the onset of a condition to constitute a viable prophylactic method or agent. Simply reducing the impact of a disease or reducing the likelihood that the disease will occur or worsen in a subject, is sufficient.

“Reducing,” “reduce,” or “reduction” means decreasing the presence, severity, or duration of at least one form of AMD, such as one or more of iAMD, NV or “wet” AMD (hereinafter simply “NV”), GA or “dry” AMD (hereinafter simply “GA”).

The term “amelioration” as used herein refers to any improvement of a disease state (for example AMD) of a patient, by the administration of one or more treatments, drugs, and/or compositions, according to the present disclosure, to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

As used herein, “subject” may be used interchangeably with “patient” and refers to a human or other mammal. A “subject in need,” includes a subject already with an existing condition (e.g., AMD), as well as a subject at risk of or susceptible to the condition and/or its progression. The term also includes human and other mammalian subjects that receive either prophylactic or therapeutic treatments as disclosed herein.

“Administration of” and “administering a” compound, composition, or agent should be understood to mean providing a compound, composition, or agent, a prodrug of a compound, composition, or agent, or a pharmaceutical composition as described herein. The compound, agent, or composition may be provided or administered by another person to the subject (e.g., infusion) or it may be self-administered by the subject.

“Intravenous” administration refers to administering a drug into a vein of a patient, e.g., by infusion (slow therapeutic introduction into the vein).

Intraperitoneal administration or injection refers to administering a drug (e.g., the disclosed peptide and/or pharmaceutically acceptable forms thereof) into the peritoneum of a patient.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. The solution can be a saline solution (e.g. about 0.9% or about 0.45% NaCl). Optionally, the IV bag is formed from polyolefin or polyvinyl chloride.

The terms “active ingredient” or “active pharmaceutical ingredient” as used herein refer to a pharmaceutical agent, active ingredient, compound, or substance, compositions, or mixtures thereof, that provide a pharmacological, often beneficial, effect.

“Pharmaceutical compositions” or “pharmaceutical formulations” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions may be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle that contributes to the desired form or consistency of the pharmaceutical composition. Each excipient or carrier must be compatible with other ingredients of the pharmaceutical composition when comingled such that interactions which would substantially reduce the efficacy of the compositions of this disclosure when administered to a subject and interactions which would result in pharmaceutical compositions that are not pharmaceutically acceptable are avoided. In addition, each excipient or carrier must be of sufficiently high purity to render it pharmaceutically acceptable. Non-limiting examples of pharmaceutically acceptable carriers may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like. In addition or alternatively, a carrier may include comprise a lubricant, a wetting agent, a flavor, an emulsifier, a suspending agent, a preservative, or the like.

The agents, compounds, compositions, antibodies, etc. used in the implementations described herein are considered to be purified and/or isolated prior to use.

The terms “dosage” or “dose” as used herein denote any form of the active ingredient formulation that contains an amount sufficient to produce a therapeutic effect with a single administration.

The term “effective amount” refers to an amount of a compound of this disclosure or other active ingredient sufficient to provide a therapeutic or prophylactic benefit in the treatment or prevention of a disease or to delay or minimize symptoms associated with a disease. Further, a therapeutically effective amount with respect to a compound means that amount of therapeutic agent alone, or in combination with other therapies, that provides a therapeutic benefit in the treatment or prevention of a disease. Used in connection with a compound of the present disclosure, the term can encompass an amount that improves overall therapy, reduces or avoids symptoms or causes of disease, or enhances the therapeutic efficacy or synergies with another therapeutic agent.

The phrase “therapeutically effective amount” means an amount of a compound of the present disclosure that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. The term “mammal” includes, but is not limited to, humans, mice, rats, guinea pigs, monkeys, dogs, cats, horses, cows, pigs, and sheep.

In this description, a “pharmaceutically acceptable salt” is a pharmaceutically acceptable, organic or inorganic acid or base salt of a compound of this disclosure. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term “prevention” as used herein means the avoidance of the occurrence or of the re-occurrence of a disease as specified herein, by the administration of an active compound to a subject in need thereof.

As used herein, the terms “protein” and “polypeptide” may be used interchangeably to designate a series of amino acid residues connected to each other by peptide bonds between the alpha-amino and carboxy groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” may be used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

MicroRNAs or “miRNAs” refer to single-stranded, non-coding RNA molecules, which may range from about 19-25 nucleotides in size.

The term “metabolite” may refer to an intermediate product produced or used during, or otherwise involved in, metabolism.

The term “specifically binds” is “antigen specific,” is “specific for,” “selective binding agent,” “specific binding agent,” “binding affinity” or is “selective” for a target refers to a peptide molecule that binds a target protein with greater affinity than other proteins, especially related proteins.

As used herein, the term “about” can mean relative to the recited value, e.g., amount, dose, temperature, time, percentage, etc., ±10%, ±9%, ±8%, ±7%, ±6%, ±5, 4%, ±3%, ±2%, or ±1%.

The singular “a,” “an,” and “the” include plural referents unless context clearly indicates otherwise. Similarly, the word “or” is intended to include “and” unless the context clearly indicates otherwise. The term “comprises” means “includes” or “contains.” Also, “comprising A or B” means including A or B, or A and B, unless the context clearly indicates otherwise. It is to be further understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

In the following sections, certain exemplary assays, systems, and methods are described in order to detail certain embodiments of this disclosure. It will be understood by one skilled in the art that practicing certain embodiments does not require the employment of all or even some of the specific details outlined herein, but rather that concentrations, times and other specific details can be modified through routine experimentation. In some cases, well known methods or components have not been included in the description.

Various systems, methods, and assays useful in determining whether a subject has AMD, including one or more specific subtypes thereof, are disclosed. The newly discovered biomarkers disclosed herein include proteins, miRNAs, and metabolites, one or more groups of which may be associated with or indicative of a particular AMD subtype, such as iAMD, NV, or GA. In accordance with embodiments disclosed herein, for instance, the presence, expression level, and/or relative expression level of one or more biomarkers may be indicative of a particular subtype or stage of AMD. For example, the presence, expression level, and/or relative expression level of proteins comprising one or more of CALL5, TPM4, CDSN, K2C1B, SSC5D, IMP4, FCN3, SLC4A1, LV746, KV621, YWHAZ, APOC4, ANT3, TTR, and/or VTN may be indicative of one or more of iAMD, NV, GA, or AMD generally. The presence, expression level, and/or relative expression level of miRNAs comprising one or more of miR-181c-5p, miR-1307-5p, miR-203a-3p, miR-107, miR-181a-5p, miR-148a-3p, miR-769-5p, miR-656-3p, miR-204-5p, miR-382-3p, miR-494-3p, miR-10399-5p, and/or miR-625-5p may be indicative of one or more of iAMD, NV, GA, or AMD generally. The presence, amount, and/or relative amount of one or more metabolites comprising one or more of L-cystine, indol pyruvate, octenoyl-l-carnitine, tetrahydrofolate, phosphoenolpyruvate, eicosapentaenoic acid, dimethylglycine, serotonin, L-citrulline, L-histidine, and/or 2-oxoglutaramate may be indicative of one or more of iAMD, NV, GA, or AMD generally.

Certain AMD subtypes may be associated with particular biomarker groups or signatures, such that the detection of one or more, including all, biomarkers present within a predefined group may be utilized to diagnose a subject with a particular form of AMD. For instance, the presence, expression level, relative expression level, amount, and/or relative amount of one or more of CALL5, TPM4, CDSN, K2C1B, SSC5D, IMP4, FCN3, miR-181c-5p, miR-1307-5p, miR-203a-3p, and/or L-cystine may be indicative of iAMD. Additional protein biomarkers indicative of iAMD may include one or more, including all, of FCN3, HBD, HPT, HV374, HV70D, KV320, KV37, 1433Z, APOA4, APOC1, APOC4, APOE, CASPE, DESP, DSG1, HORN, K1C10, K1C14, K1C16, K1C9, K22E, K2C1, K2C5, K2C6A, K2C6B, KPRP, PDIA3, PLAK, S10A7, SRCRL, THIO, TPM4, XP32, and/or ZA2G. In some embodiments, one or more proteins, including all, of the following may be expressed significantly higher in healthy individuals relative to subjects with iAMD: FCN3, HBD, HPT, HV374, HV70D, KV320, and KV37. In some embodiments, one or more proteins, including all, of the following may be expressed significantly higher in iAMD subjects relative to healthy individuals: 1433Z, APOA4, APOC1, APOC4, APOE, CALL5, CASPE, CDSN, DESP, DSG1, HORN, K1C10, K1C14, K1C16, K1C9, K22E, K2C1, K2CIB, K2C5, K2C6A, K2C6B, KPRP, PDIA3, PLAK, S10A7, SRCRL, THIO, TPM4, XP32, and ZA2G.

Additional miRNA biomarkers indicative of iAMD may include one or more, including all, of hsa-miR-100-5p, hsa-miR-107, hsa-miR-127-3p, hsa-miR-1307-5p, hsa-miR-181c-5p, hsa-miR-203a-3p, and hsa-miR-205-5p, which may be expressed significantly higher in iAMD patients relative to healthy individuals. In some embodiments, hsa-miR-486-3p may be expressed significantly higher in healthy individuals relative to subjects with iAMD.

The presence, expression level, relative expression level, amount, and/or relative amount of one or more of miR-107, miR-181a-5p, miR-148a-3p, and/or miR-769-5p may be indicative of AMD as a whole.

The presence, expression level, relative expression level, amount, and/or relative amount of one or more of SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and/or 2-oxoglutaramate may be indicative of NV. Additional protein biomarkers indicative of NV may include one or more, including all, of B3AT, FCN3, HBD, HV102, HV313, HV374, HV70D, IGHA2, KV116, KV621, LV211, and LV746. In some examples, one or more of such proteins may be expressed significantly higher in healthy individuals than subjects with NV.

In some examples, additional miRNAs indicative of NV may include one or more, including all, of: hsa-miR-100-5p, hsa-miR-382-3p, hsa-miR-204-5p, and hsa-miR-656-3p, which may be expressed significantly higher in subjects with NV relative to healthy individuals.

The presence, expression level, relative expression level, amount, and/or relative amount of one or more of SSC5D, YWHAZ, APOC4, ANT3, TTR, VTN, miR-494-3p, miR-10399-5p, miR-625-5p, and/or phosphoenolpyruvate may be indicative of GA. Additional protein biomarkers indicative of GA may include one or more, including all, of A1BG, ALBU, ANT3, CBPN, CPN2, KLKB1, HBD, HRG, HV102, IGHA2, VTNC, HV70D, MBL2, PLMN, TTHY, 1433Z, FILA2, K1C14, APOC4, KV117, and/or SRCRL. In some embodiments, one or more proteins, including all, of the following may be expressed significantly higher in healthy individuals relative to subjects with GA: A1BG, ALBU, ANT3, CBPN, CPN2, KLKB1, HBD, HRG, HV102, IGHA2, VTNC, HV70D, MBL2, PLMN, and TTHY. In some embodiments, one or more proteins, including all, of the following may be expressed significantly higher in GA subjects relative to healthy individuals: 1433Z, FILA2, K1C14, APOC4, KV117, and SRCRL.

Additional miRNA biomarkers indicative of GA may include one or more, including all, of: hsa-niR-125b-5p, hsa-miR-1301-3p, hsa-miR-181a-5p, hsa-miR-30a-5p, hsa-miR-30b-5p, hsa-miR-345-5p, hsa-miR-10b-5p, hsa-miR-125a-5p, hsa-miR-99a-5p, hsa-miR-10399-5p, hsa-miR-107, hsa-miR-148a-3p, hsa-miR-152-3p, hsa-miR-199a-3p, hsa-niR-199b-3p, hsa-miR-30e-5p, hsa-miR-382-3p, hsa-miR-494-3p, hsa-miR-625-5p, hsa-miR-92a-3p, and hsa-miR-30d-5p. In some examples, one or more miRNAs, including all, of the following may be expressed significantly higher in healthy individuals relative to subjects with GA: hsa-miR-125b-5p, hsa-miR-1301-3p, hsa-miR-181a-5p, hsa-miR-30a-5p, hsa-miR-30b-5p, hsa-miR-345-5p, hsa-miR-10b-5p, hsa-niR-125a-5p, and hsa-miR-99a-5p. In some examples, one or more miRNAs, including all, of the following may be expressed significantly higher in subjects with GA relative to healthy individuals: hsa-miR-10399-5p, hsa-miR-107, hsa-miR-148a-3p, hsa-miR-152-3p, hsa-miR-199a-3p, hsa-miR-199b-3p, hsa-niR-30e-5p, hsa-miR-382-3p, hsa-miR-494-3p, hsa-miR-625-5p, hsa-miR-92a-3p, and hsa-miR-30d-5p.

In some embodiments, the presence, expression level, relative expression level, amount, and/or relative amount of one or more proteins, miRNAs, and/or metabolites in one subject with an AMD condition may be indicative of an AMD condition in a different subject. For instance, hsa-miR-18a-5p and/or hsa-miR-625-5p may be expressed significantly higher in GA patients versus iAMD patients. In some examples, one or more of hsa-let-7c-5p, hsa-miR-100-5p, hsa-miR-125b-5p, hsa-miR-99a-5p, and hsa-miR-205-5p may be expressed significantly higher in iAMD patients relative to GA patients. In additional examples, one or more of hsa-miR-136-3p, hsa-miR-1843, hsa-miR-18a-5p, hsa-miR-200b-3p, hsa-miR-454-5p, hsa-miR-4677-3p, hsa-miR-625-5p, and hsa-miR-629-5p may be expressed significantly higher in GA patients versus NV patients. In some examples, one or more of hsa-miR-25-3p, hsa-miR-17-5p, hsa-miR-199a-3p, hsa-miR-320a-3p, letc-5p, and hsa-miR-125b-5p may also be expressed significantly higher in GA patients versus NV patients. In some examples, one or more of hsa-miR-27b-3p, hsa-miR-100-5p, hsa-miR-122-5p, hsa-miR-204-5p, hsa-miR-199b-5p, hsa-miR-151a-3p, and hsa-miR-125a-3p may be expressed significantly higher in NV patients versus GA patients.

Accordingly, assays or methods of determining whether a subject has AMD, or more specifically iAMD, NV and/or GA, may involve determining and/or measuring the presence, expression level, relative expression level, amount, or relative amount of one or more of the aforementioned biomarkers present within a biological sample obtained from the subject, and comparing the determined/measured values to one or more corresponding reference values or thresholds, which may be derived from healthy individuals or subjects with a different form of AMD relative to the subject being tested.

Subjects from which the biological samples are obtained may include subjects suspected of having AMD, such as subjects experiencing vision loss. Subjects may also include healthy subjects experiencing no abnormal vision changes. Subjects may also include healthy subjects of at least 50 years of age, who may be tested at a regular medical check-up, for instance, as they may be at a heightened risk of developing AMD, as are subjects with a family history of AMD.

The biological sample may be collected via non-invasive means at a point of care. Embodiments of the biological sample include a biofluid, which may include or consist of plasma. Additional embodiments of the biological sample include or consist of one or more other biofluids, such as urine, tears, saliva, aqueous humor, vitreous, or serum. Accordingly, assays or methods of AMD assessment or diagnosis may involve obtaining, via fluid biopsy, a biological sample from a subject, which may be stored in one or more vials. In embodiments utilizing plasma, the plasma may be collected at least in part via centrifugation following phlebotomy. Vials of biological samples and components thereof may be stored in a cooling apparatus, e.g., a freezer.

The presence, expression level, relative expression level, amount, and/or relative amount of one or more of the aforementioned biomarkers present within the biological sample may be determined, in part or exclusively, from the cargo contained within EVs derived and isolated from the biological sample. Capturing and isolating the EVs present within a biological sample may involve size exclusion chromatography in some embodiments. Embodiments may additionally or alternatively include the use of ultracentrifugation and/or density gradient separation. Examples may also involve confirming that EVs have indeed been isolated and purified with specificity by assessing the morphology, molecular composition, size, and/or distribution of the vesicles, for example via one or more of electron microscopy (TEM), immunoblotting, and NanoSight.

After collecting a biological sample and isolating the EVs therefrom, the proteins, miRNAs, and/or metabolites present within the EVs may be harvested and identified. In some examples, total protein, miRNA, and/or metabolite content may be obtained and compared to one or more corresponding reference levels defined by one or more biomarker panels, which may include the aforementioned biomarkers associated with one or more of iAMD, NV, and GA. Examples may additionally or alternatively involve a targeted approach that involves specifically determining the presence, expression level, relative expression level, amount, and/or relative amount of only one or more of the aforementioned biomarkers, for example via Western blotting, mass spectroscopy, RNA sequencing, and/or metabolomics protocols. One or more variations of ECL, ELISA, Ella, Surface Plasmon Resonance, molecular array, SIMOA, PCR, and/or qPCR may also be used. Relative amounts and/or expression levels may be determined by comparing measured values (e.g., protein expression levels, miRNA expression levels, metabolite contents, etc.) to one or more reference levels, which may be considered or presented in the form of threshold values.

The relative amounts and/or expression levels of the at least one biomarker may be greater or lesser than a reference. In various embodiments, the biomarker amount and/or expression level may be increased or decreased from more than about 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 55×, 60×, 65×, 70×, 75×, 80×, 85×, 90×, 95×, or 100× to less than about 105×, 105×, 100×, 95×, 90×, 85×, 80×, 75×, 70×, 65×, 60×, 55×, 50×, 45×, 40×, 35×, 30×, 25×, 20×, 19×, 18×, 17×, 16×, 15×, 14×, 13×, 12×, 11×, 10×, 9×, 8×, 7×, 6×, 5×, 4×, 3×, or 2×. In some examples, the biomarker amount and/or expression level may be zero or approximately zero when compared to one or more, or all, AMD types and/or healthy individuals.

In some embodiments, at least one biomarker indicative of a certain AMD subtype must be detected to diagnose a subject with that AMD subtype. In other embodiments, at least two, three, four, five, six, seven, eight, nine, ten, 11, 12, 13, 14, or 15 biomarkers, or more, indicative of a certain AMD subtype must be detected to diagnose a subject with that subtype. Generally, the accuracy of a particular AMD diagnosis may increase as the number of biomarkers associated with that subtype are detected. In some examples, the detection of certain biomarkers may more indicative of the presence of the disease than other biomarkers, even though both may be significant.

Based on the detection of one or more biomarkers present within a biological sample, the subject from which the sample was obtained may be diagnosed with one or more forms of AMD, including iAMD, NV, and/or GA. Embodiments may also involve determining a likelihood of the subject developing a more severe or advanced form of the disease. For instance, the presence of one or more biomarkers of iAMD may indicate a moderate to high likelihood that the subject will, in the future, develop NV and/or GA, as patients with iAMD are at risk of developing such visually threatening forms of advanced AMD. In this regard, “at-risk” patients may be identified.

In some examples, the biomarker testing results, including any corresponding determinations or diagnoses, may be presented on a customized display on a graphical user interface. The display may include testing results obtained over time, along with a risk score related to the likelihood of a patient developing a more advanced form of AMD.

### Therapeutic Approaches

AMD is a degenerative condition of the photoreceptors, which are the cells in the retina that respond to light, and the pigment epithelial cells that support their function. This degenerative process targets central vision, leading to loss of the sharp fine-detail vision required for activities like reading, driving, and recognizing faces. The biomarker-based methods of diagnosing, monitoring, and predicting AMD progression disclosed herein may enable early intervention therapeutic approaches that stop, slow, or even reverse AMD, thereby improving quality of life.

A treatment regimen may be determined in view of the biomarker testing results. In some examples, the treatment regimen may follow established protocols for AMD patients, or subjects identified as being high-risk. Such treatments may be modified based in view of the biomarkers identified. Advantageously, the disclosed systems may enable early diagnosis of one or more AMD subtypes relative to preexisting approaches, allowing treatment to begin sooner and thereby increasing the likelihood of successfully treating the condition. Treatments may include, among other things, the administration, for example via eye injection, a therapeutically effective amount of one or more pharmaceutical compositions, depending on the AMD diagnosis and/or likely disease progression. Photodynamic therapy and/or laser therapy may additionally or alternatively be implemented, also depending on the AMD diagnosis and/or likely disease progression. Various nutritional supplements can also be taken by the subject, including those containing vitamin C and/or E, lutein, zinc, copper, and/or zeaxanthin. A variety of medications can also be prescribed, including one or more of Aflibercept, Ranibizumab, Bevacizumab, Faricimab-svoa, and Brolucizumab.

In various embodiments, different therapeutic approaches may be implemented for subjects diagnosed with different AMD types. For example, subjects diagnosed with iAMD in accordance with the methods described herein may be administered a variety of nutritional supplements aimed at slowing the progression of the disease. Antioxidant vitamins and minerals, including vitamin C, vitamin E, zinc, copper, lutein, and zeaxanthin may be provided. The Age-Related Eye Disease Study (“AREDS”) and AREDS2 formulations may also be provided for individuals with iAMD and GA. Lifestyle modifications may also be recommended. For example, regular exercise, balanced diets, and low exposure to UV light may reduce the risk of vision loss by 70%. For NV patients, detection of one or more of the biomarkers disclosed herein may also inform the selection of a particular treatment, which may include intravitreal injections of anti-vascular endothelial growth factor (VEGF) drugs such as ranibizumab (Lucentis), aflibercept (Eylea), and bevacizumab (Avastin), which may reduce abnormal blood vessel growth and leakage, thereby slowing down disease progression and preserving vision. Embodiments of NV treatment may also involve photodynamic therapy (PDT), which may involve injecting a light-sensitive drug into the bloodstream, which is then activated by laser light to destroy abnormal blood vessels in the retina. Embodiments of NV treatment may also involve laser therapy, including focal laser photocoagulation and thermal laser treatment, to seal off abnormal blood vessels or cauterize leaking vessels. Embodiments of NV treatment may also involve intraocular steroid injections to reduce inflammation and fluid accumulation in the retina.

In some examples, GA treatments may include the administration of a complement C5 inhibitor, e.g., Izervay (avacincaptad pegol). Additional therapies may include other complement inhibitors, anti-inflammatories, visual cycle modulators, and cell-based therapies.

Embodiments of administered pharmaceutical compositions may include, or be administered concurrently with, at least one pharmaceutically acceptable carrier. Relatedly, the pharmaceutical composition may be administered singly or in combination with other therapeutic agents, either serially or simultaneously. Such additional agents may or may not be formulated to treat the same conditions.

The pharmaceutical compositions disclosed herein may be administered using an infusion system or device, or an injection device, such as tuberculin syringe, intravitreal injection device, or IV drip device, which may be configured specifically for the purposes described herein. In some examples, the administration device may be a single-use device, which may be included in a kit that also includes a single dose of a pharmaceutical composition. Accordingly, an injection device may constitute a part of a system for treating, reducing the risk of, preventing, or alleviating at least one symptom of AMD.

To accommodate multiple administration techniques and schedules, one or more pharmaceutical compositions may be prepared in a unit-dosage form or multiple-dosage form, along with a pharmaceutically acceptable carrier and/or excipient according to a method employed by those skilled in the art. Example formulations may be in the form of an aqueous or oil-based solution, a suspension, or an emulsion. For increased stability and long-term storage, the pharmaceutical compositions may be lyophilized. Pharmaceutical compositions, including those taken orally, applied onto the surface of the eyes, or injected intravitreally may be administered on a regular basis, e.g., daily, for a treatment period, which may end at a predefined time point or after symptoms subside or disappear.

Improved patient outcomes achieved via the disclosed methods may include the inhibition of AMD, including one or more symptoms thereof, e.g., vision loss. Progression of the disease may be slowed, stopped, or reversed in patients subjected to the biomarker testing and subsequent treatment disclosed herein relative to patients not subjected to the biomarker testing and subsequent treatment.

### Kits

This disclosure further relates to a kit comprising a pharmaceutical composition (or compositions) for use in a method of treating or alleviating a symptom of AMD. In certain embodiments, kits are provided for storage, transport and use in treating or alleviating a target disease, such as one or more forms of AMD, as described herein. In some embodiments, kits can include one or more containers, e.g., vials, and syringes.

### EXAMPLES

The following examples are included to illustrate certain embodiments. It should be appreciated by those of skill in the art that the techniques disclosed in the examples represent techniques discovered to function well in the practice of the claimed methods, compositions and systems. However, those of skill in the art should, in light of the present disclosure, appreciate that changes can be made in some embodiments which are disclosed and still obtain a like or similar result without departing from the spirit and scope of embodiments disclosed herein.

The following examples evaluated the profiles of circulating markers in plasma by determining and characterizing the cargo contained in plasma-derived exosornes or extracellular vesicles (hereinafter “EVs”), including the proteome and transcriptome cargo, in patients with different phenotypes of AMD. As further set forth below, the experimental results showed that certain biomarkers distinguished patients with and without iAMD, including patients with iAMD who progressed to advanced AMD. Among patients with iAMD, a biomarker profile identified via implementation of the experiments below identified patients who will likely convert to advanced AMD. Accordingly, with the data collected, subjects at a high risk for developing severe AMD and progressive visual loss can be identified early, which can inform the development of targeted interventions to interrupt the natural progression of AMD.

Plasma metabolome samples were collected from ten patients with bilateral neovascular AMD (hereinafter “NV”), iAMD, and bilateral GA (hereinafter “GA”). Cases of advanced AMD were age-matched to ten healthy individuals or controls without AMD. iAMD was defined as the presence of large drusen (>125 m) and/or any AMD pigmentary abnormalities in the macula. NV was defined as the presence of choroidal neovascularization based on OCT. GA was defined as circumscribed areas of atrophy, which reflect cell death in the RPE, outer retina, and choriocapillaris in patients with AMD, but no other retinal disease. Diagnoses of iAMD, NV, and GA were confirmed using multimodal imaging (FAF, NIR, and OCT).

### Plasma Sample Collection

Following phlebotomy, each plasma sample was isolated via centrifugation, implemented in a tube of EDTA at 3000 revolutions per minute for 10 minutes in a cooled centrifuge. The plasma was transferred into another tube for a similar second spin. The second supernatant was aliquoted into cryovials of 250 to 500 μL, depending on the sample volume available. All samples were stored at −80° C.

### Extracellular Vesicle (EV) Isolation

To purify the EVs, size exclusion chromatography was performed using SmartSEC Single for EV Isolation (System Biosciences) according to the manufacturer's instructions. Starting amounts were 250 microliters per patient, with addition of PBS according to protocol. EV concentration and size were assessed using the NanoSight NS500 system. Based on the NanoSight protocol, to ensure accurate readings, samples were diluted at 1:50 and in 1 ml of filtered PBS to ensure a representative graph of the sample (compared across five trials). The NanoSight system uses a laser light source to illuminate nano-scale particles, detected individually as light-scattered points moving via Brownian motion. The polydispersity was quantified, and Nanoparticle Tracking Analysis (NTA) software 2.3 was used to track the size and diffusion of nanoparticles. Results are displayed as a frequency size distribution graph describing the number of particles per ml.

EV morphology, molecular composition, size, and distribution were characterized by electron microscopy (TEM), immunoblotting, and NanoSight. Typical rounded membrane vesicles within a size range of 30-170 nm were observed. The physical characteristics of the vesicle preparations and their biochemical composition (presence of Syntenin and absence of GM130) confirmed that the EVs fulfilled the criteria for EVs.

### Mass Spectrometry (EV Proteins)

Samples were subjected to proteolytic digestion using a filter-aided sample preparation (FASP) protocol (Wisniewski, J. R., Zougman, A., Nagaraj, N. & Mann, M. Universal sample preparation method for proteome analysis. Nat Methods 6, 359-362 (2009)) with 10 kDa molecular weight cutoff filters (Sartorius Vivacon 500 #VN01H02). Samples were reduced with 5 mM tris (2-carboxyethyl)phosphine), alkylated with 50 mM 2-chloroacetanmide, and digested overnight with trypsin (enzyme:substrate ratio 1:50) at 37° C. Peptides were recovered from the filter using successive washes with 0.2% formic acid (FA). Aliquots containing 10 μg of digested peptides were cleaned using Pierce™ C18 Spin Tips (Thermo Scientific) according to the manufacturer's protocol, dried in a vacuum centrifuge, and resuspended in 0.1% FA in mass spectrometry-grade water. Digested peptides were loaded onto Evotips and analyzed directly using an Evosep One liquid chromatography system (Evosep Biosystems, Denmark) coupled with a Bruker timsTOF SCP mass spectrometer (Bruker, Germany). Peptides were separated on a 75 μm i.d.×15 cm separation column packed with 1.9 μm C18 beads (Evosep Biosystems, Denmark) and over a predetermined 44-minute gradient. Buffer A was 0.1% FA in water and buffer B was 0.1% FA in acetonitrile. Instrument control and data acquisition were performed using Compass Hystar (version 6.0) with the timsTOF SCP operating in parallel accumulation-serial fragmentation (PASEF) mode under the following settings: mass range 100-1700 nm/z, 1/k/0 Start 0.7 V s cm-2 End 1.3 V s cm-2; ramp accumulation times were 166 ms; capillary voltage was 4500 V, dry gas 8.0 L min-1 and dry temp 200° C. The PASEF settings were: 5 MS/MS scans (total cycle time, 1.03 s); charge range 0-5; active exclusion for 0.2 min; scheduling target intensity 20,000; intensity threshold 500; collision-induced dissociation energy 10 eV. Fragmentation spectra were searched against the UniProt human proteome database (Proteome ID #UP000005640) using the MSFragger-based FragPipe computational platform (Kong, A. T., Leprevost, F. V., Avtonomov, D. M., Mellacheruvu, D. & Nesvizhskii, A. I. MSFragger: ultrafast and comprehensive peptide identification in mass spectrometry-based proteomics. Nat Methods 14, 513-520 (2017)). Contaminants and reverse decoys were added to the database automatically. The precursor-ion mass tolerance and fragment-ion mass tolerance were set to 10 ppm and 0.2 Da, respectively. Fixed modifications were set as carbamidomethyl (C), and variable modifications were set as oxidation (M) and two missed tryptic cleavages were allowed, and the protein-level false discovery rate (FDR) was <1%.

### Proteomic Profiling Analysis (EV Proteins)

Data analysis was conducted in R (version 4.2.0) and MetaboloAnalyst (Xia, J., Psychogios, N., Young, N. & Wishart, D. S. MetaboAnalyst: a web server for metabolomic data analysis and interpretation. Nucleic Acids Res 37, W652-660 (2009)). Proteins found in ⅔ of the samples in at least one group were selected for further analysis. Protein intensities were input into Metaboanalyst software for analysis with the Statistical Analysis-one factor module applying log 10 transformation and sample normalization by sum as settings, and missing values were replaced by LoDs (⅕ of the minimum positive value of each variable). ANOVA followed by a Fisher's posthoc test was used to obtain those proteins showing statistical different expression between groups. Proteins with adjusted p-value <0.05 were subjected to hierarchical clustering within the heatmaps (ComplexHeatmap package) using ward. D2 clustering method (hclust function). Gene set enrichment analysis was performed using Gene Ontology Biological Process, Cellular Component, and Molecular Function terms (https://maavanlab.cloud/Enrichr/) using proteins found in extracellular vesicles. Differential expression proteins (p<0.05) between healthy individuals and AMD patient groups were performed using unpaired t-tests. p values and log 2 fold changes were calculated and shown as volcano plots. Data visualization was performed using ggplot2 and Circlize packages.

Small RNA Library Preparation and Sequencing (EV miRNAs)

For NGS of EVs, small RNA Libraries were generated using a NEBNext Multiplex Small RNA Library Prep Set for Illumina (NEB, Ipswich, MA). The Multiplex Small RNA Library prep set has an input concentration range and does not rely on starting the library using an equal RNA amount. Next, three biological and three technical replicates were used for each EV experimental sample. Libraries were prepared according to the manufacturer's instructions. The amplified libraries were resolved on Novex 6% TBE Gels (Thermo Fisher Scientific, Waltham, MA). Library fragments were excised and further purified using a RNA Clean-up and Concentration Micro-Elute Kit (Norgen Biotek, Thorold, Ontario). The indexed libraries were quantified using the High Sensitivity DNA Kit (Agilent, Santa Clara, CA). All libraries derived from EVs were pooled together for a final library at a final concentration of 4 nM. Normalization of libraries after size selection is the used method for small RNA sequencing. The libraries were pooled using equal molarities from the NGS library of each sample. The pooled library was then sequenced on an Illumina MiSeq using version 3 reagents by Norgen Biotek Corp. A small RNA expression profile was generated from the resulting FASTQ files using the exceRpt small RNA-seq pipeline. Sequencing was quality controlled using a Nanodrop ND/1000 spectrophotometer. Library QC was performed through the quantification of the indexed libraries using the High Sensitivity DNA Kit (Agilent, Santa Clara, CA).

RNA Seq Analysis (EV miRNAs)

Small RNAseq data was processed through the Galaxy platform. First, raw sequence reads were trimmed to remove adapters using Trim Galore8 and quality visualization was performed using FastQC. Sequences were mapped to mature miRNAs within the miRBase v.22 database and counts were quantified with miRDeep2 program using default settings (Mackowiak, S. D. Identification of novel and known miRNAs in deep-sequencing data with miRDeep2. Curr Protoc Bioinformatics, Chapter 12, 12 10 11-12 10 15 (2011)). Downstream data analysis was conducted in R (version 4.2.0). Principal component analysis (PCA) was performed on the total miRNA profile, and abundance of miRNAs were quantified as the percentage of the total counts in each sample. Differential expression analysis was performed using DESeq2. miRNAs with p-value <0.05 were labeled as differentially expressed miRNAs. ComplexHeatmap R package was used to visualize differential expression of specific miRNAs. For identification of predicted targets of the differentially expressed miRNAs, proteins found in Retina and RPE tissue proteomics data were used to obtain miRNA-target interactions according to IPA microRNA Target Filter tool (Kramer, A., Green, J., Pollard, J., Jr. & Tugendreich, S. Causal analysis approaches in Ingenuity Pathway Analysis. Bioinformatics 30, 523-530 (2014)). Different miRNA target prediction programs (TargetScan, miRecords, Ingenuity Knowledge Base, and TarBase) filtered the miRNA-target pairings. Confidence filter was used by selecting both experimentally observed and high predicted target pairings. Gene set enrichment analysis of obtained targets were performed using Gene Ontology Biological Process terms.

### Ultra-High-Performance Liquid Chromatography Tandem Mass Spectrometry (UHPLC-MS) Metabolomics (EV Metabolites).

All solvents were Optima grade (Fisher Scientific). EV samples were thawed on ice, then 25 μL of EVs were mixed with 50 μL of ice-cold extraction buffer (5:3:2 MeOH/ACN/H2O). For absolute quantification, all stable isotope-labeled standards were purchased from Cambridge Isotope Laboratories. Where applicable, the extraction buffer contained 3.75 μM of an amino acid mixture (MSK-A2-1.2), an acylcarnitine mixture (NSK-B) diluted 1:200 according to the manufacturer's instructions (final concentrations: free carnitine D9, 0.76 nM; acetylcarnitine D3, 0.19 nM; propanoyl D3, 0.038 nM; butyryl D3, 0.038 nM; isovaleryl D9, 0.038 nM; octanoyl D3, 0.038 nM; myristoyl D9, 0.038 nM; palmitoyl D3, 0.076 nM), [2,2,4,4-D4]citrate (1.5 μM), [U-13C]α-ketoglutarate (1.5 μM), [U-13C]succinate (1.5 μM), [1,4-13C2]fumarate (1.5 μM), and [1-13C]pyruvate (1.5 μM). Samples were vortexed at 4° C. for 30 min, then spun for 10 min at 10,000 g at 4° C. Protein and lipid pellets were discarded, and supernatants were analyzed by ultra-high-pressure liquid chromatography-mass spectrometry (UHPLC-MS) on a Thermo Vanquish UHPLC (San Jose, CA) coupled to a Thermo Q Exactive mass spectrometer (Bremen, Germany) in positive and negative ion modes (separate runs). Solvents were water and acetonitrile supplemented with formic acid (0.1%—positive mode) or ammonium acetate (5 mM—negative mode). Initial analysis utilized a 3-min isocratic run; subsequent extensive analysis (including absolute quantification) was performed using a 9-minute gradient from 5% to 95% acetonitrile organic phase. Samples were randomized, and a quality-control sample was injected every 10 runs. Data analysis was performed using Maven (Princeton University) following file conversion by MassMatrix (Case Western Reserve University). Absolute concentrations were obtained using the following equation: light (peak area/peak area) heavy DF light heavy [ ]=[ ]*where DF is the dilution factor, in this case, 3 (i.e., 25 μL of EVs in a total 75 μL volume). Absolute concentrations for additional acylcarnitines were estimated using the labeled acylcarnitine with closest structural similarity (i.e., similar fatty acyl moiety carbon backbone length). Relative quantification data were normalized to median and autoscaled within the MetaboAnalyst 3.0 platform prior to visualization and statistical analysis. Hierarchical clustering analysis was performed using GENEE (Broad Institute). Bar graphs were prepared using GraphPad Prism 5.03. Receiver operating characteristic curves, partial least-squares-discriminant analysis, and statistical analysis (ANOVA) for heat maps prepared using MetaboAnalyst 3.0.

### Statistical Analysis

Statistical analyses were performed using GraphPad Prism software and shown as mean±SD. Three biological replicates were used for analysis in all cases. The statistical significance of the difference was determined using Student t-test, and the one-way ANOVA with Tukey post-test conducted for multiple comparisons. Significant differences were denoted with asterisks: *(p<0.05), **(p<0.01), ***(p<0.001), ****(p<0.0001). Lipidomic and Metabolomic expression analysis are described in their corresponding sections.

### Results

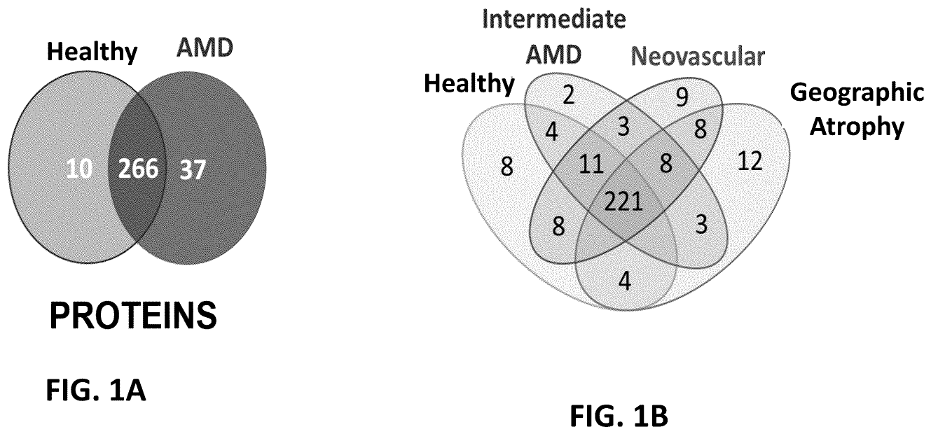

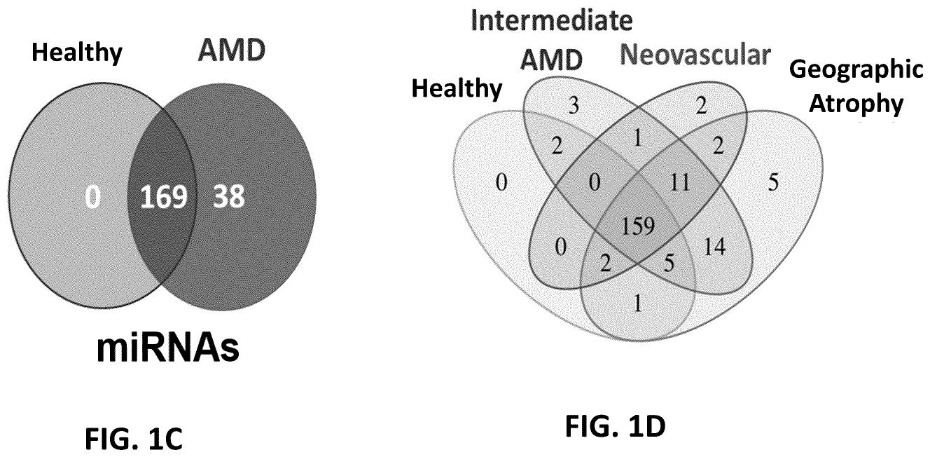

As noted above, purified EVs from plasma were subjected to mass spectroscopy. The Venn diagram of FIG. 1A was generated using the results of that analysis, showing total detectable spectral protein counts found in healthy individuals versus all AMD types, i.e., iAMD, NV, and GA. As shown, 10 proteins were specific to the healthy control, 37 proteins were specific to AMD patients, and 266 proteins overlapped between the two groups. FIG. 1B is a Venn diagram showing proteins overlapping and differing between healthy individuals compared to patients with each of iAMD, NV, and GA. As shown, eight proteins were specific to the healthy control, 12 proteins were specific to patients with GA, nine proteins were specific to patients with NV, and two proteins were specific to patients with iAMD. Of the 319 total proteins identified, 221 overlapped between all groups.

FIG. 1C is a Venn diagram showing total detectable miRNAs found in healthy individuals versus all AMD conditions, i.e., iAMD, NV, and GA. The results showed that zero miRNAs were specific exclusively to the healthy control, 38 miRNAs were specific to AMD patients, and 169 miRNAs overlapped between the two groups. FIG. 1D is a Venn diagram showing miRNAs overlapping and differing between healthy individuals compared to patients having iAMD, NV, and GA. As shown, zero miRNAs were specific to the healthy control, five miRNAs were specific to patients with GA, two miRNAs were specific to patients with NV, and three miRNAs were specific to patients with iAMD. 159 miRNAs overlapped between all groups.

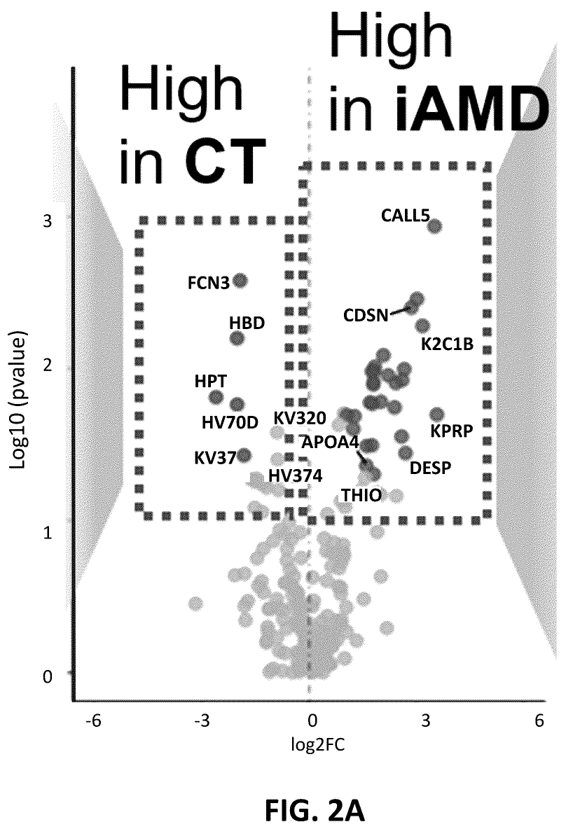

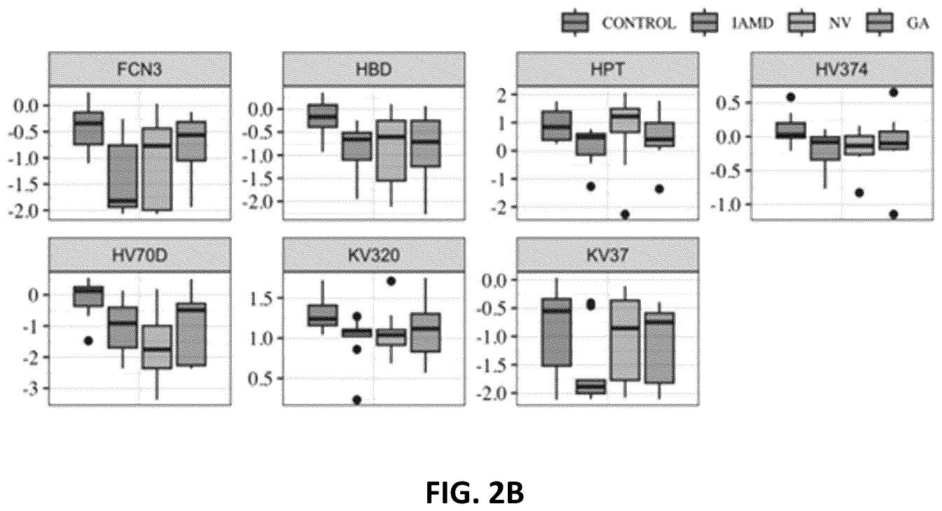

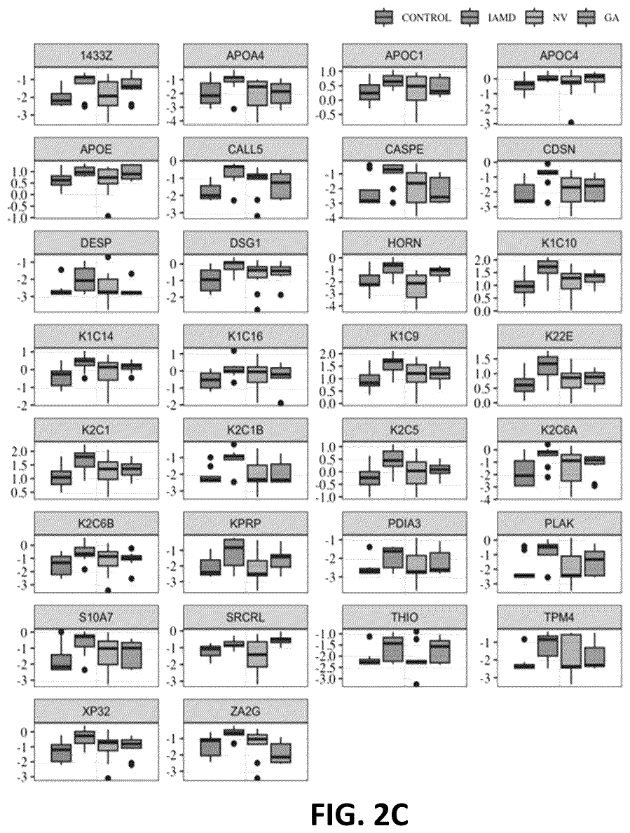

FIGS. 2A-2C depict protein expression in healthy individuals versus subjects afflicted with iAMD. In particular, FIG. 2A is a volcano plot highlighting the proteins expressed highly in the control individuals versus iAMD patients, including but not limited to FCN3, HBD, HPT, HV70D, KV37, CALL5, TPM4, CDSN, K2C1B, KPRP, POA4, and DESP. The diagram of FIG. 2B shows the seven proteins expressed significantly higher in healthy individuals (CT) compared to iAMD patients, namely: FCN3, HBD, HPT, HV374, HV70D, KV320, and KV37. The diagram of FIG. 2C shows the proteins expressed significantly higher in iAMD patients compared to healthy individuals, namely: 1433Z, APOA4, APOC1, APOC4, APOE, CALL5, CASPE, CDSN, DESP, DSG1, HORN, K1C10, K1C14, K1C16, K1C9, K22E, K2CL, K2C0B, K2C5, K2C6A, K2C6B, KPRP, PDIA3, PLAK, S10A7, SRCRL, THIO, TPM4, XP32, and ZA2G.

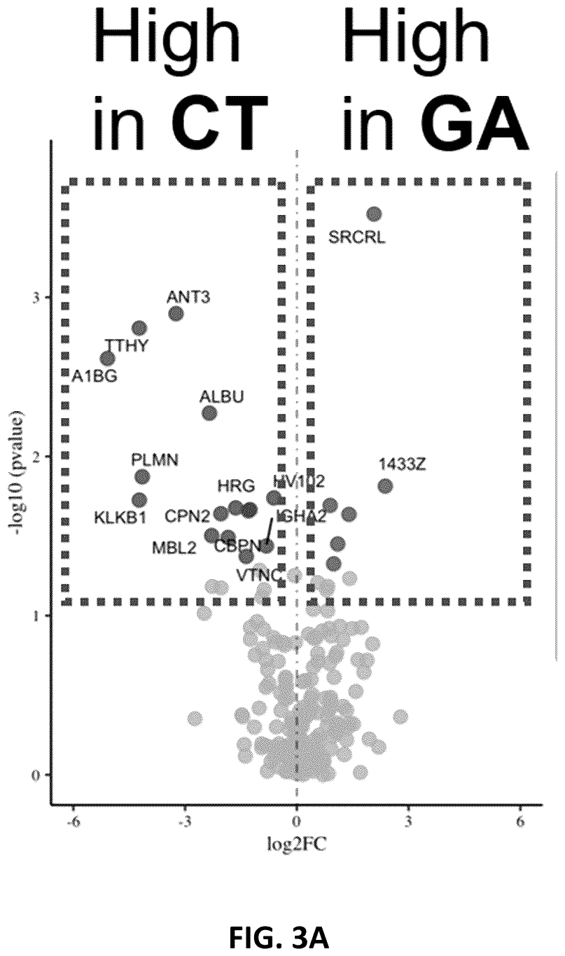

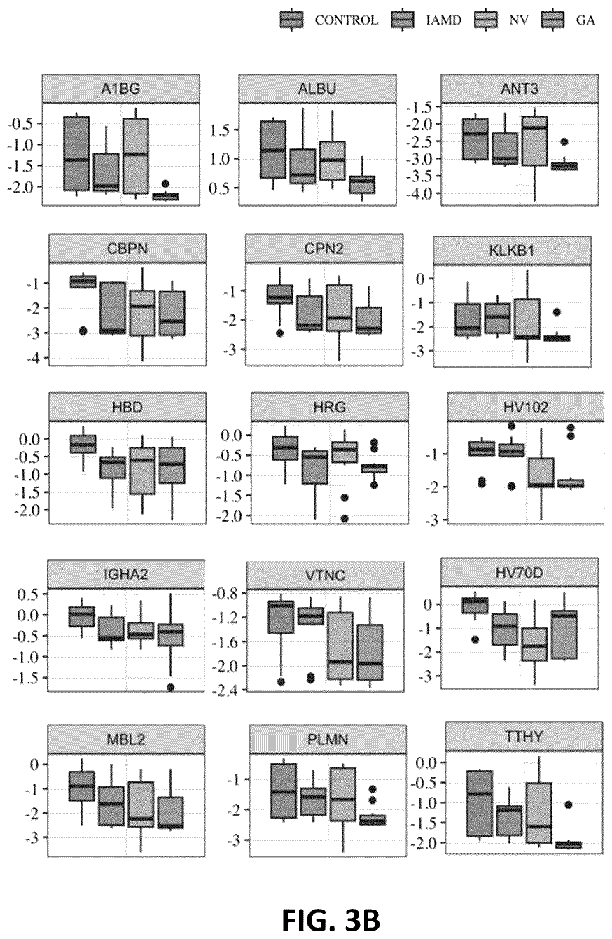

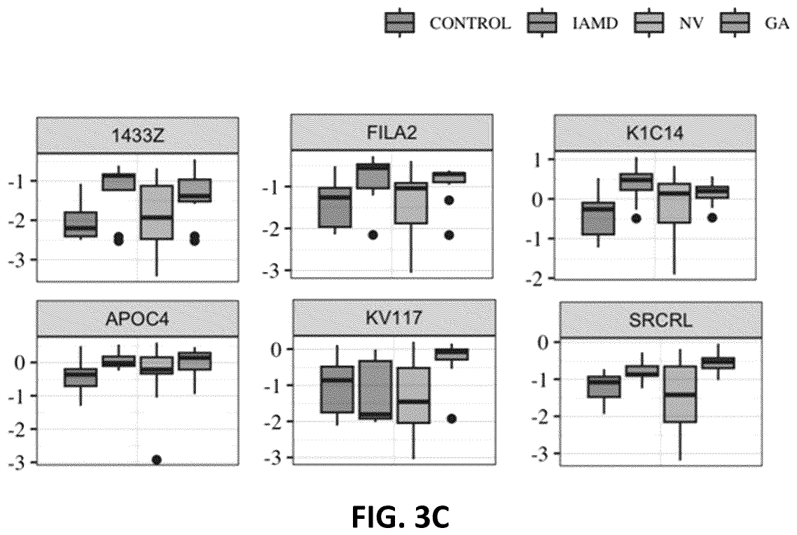

FIGS. 3A-3C depict protein expression in healthy individuals versus subjects afflicted with GA. In particular, FIG. 3A is a volcano plot highlighting the proteins expressed highly in the control individuals versus the GA patients, including but not limited to ANT3, TTHY, A1BG, ALBU, PLMN, KLKB1, CPN2, HRG, MBL2, CBPN, VTNC, 1433Z, AND SRCRL. The diagram of FIG. 3B shows the 15 proteins expressed significantly higher in healthy individuals (CT) compared to GA patients, namely: A1BG, ALBU, ANT3, CBPN, CPN2, KLKB1, HBD, HRG, HV102, IGHA2, VTNC, HV70D, MBL2, PLMN, and TTHY. The diagram of FIG. 3C shows the six proteins expressed significantly higher in GA patients compared to healthy individuals, namely: 1433Z, FILA2, K1C14, APOC4, KV117, and SRCRL.

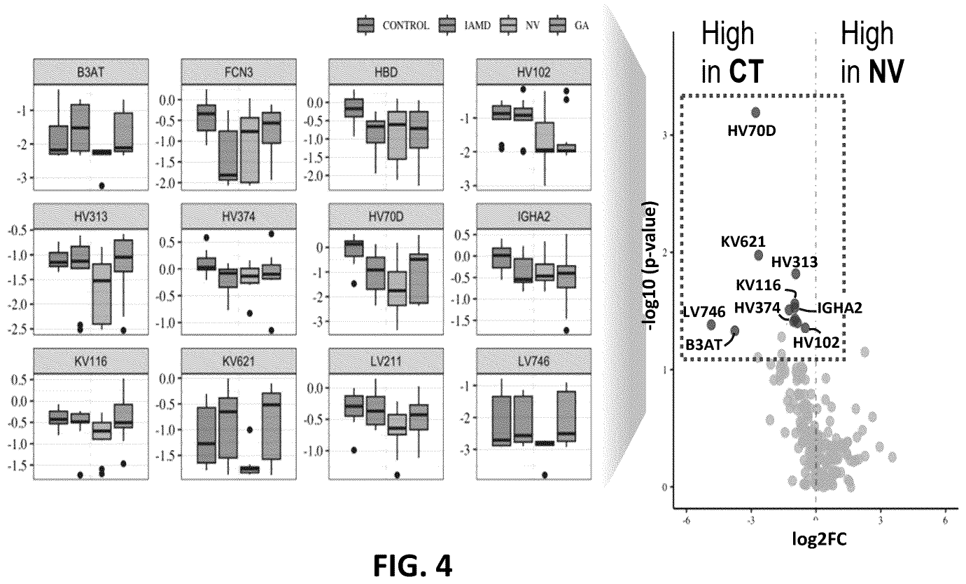

FIG. 4 depicts protein expression in healthy individuals versus NV patients, including a volcano plot highlighting the proteins expressed highly in the control individuals versus the NV patients, and a diagram (left) showing the 12 proteins expressed significantly higher in healthy individuals (CT) than NV patients, namely: B3AT, FCN3, HBD, HV102, HV313, HV374, HV70D, IGHA2, KV116, KV621, LV21I, and LV746.

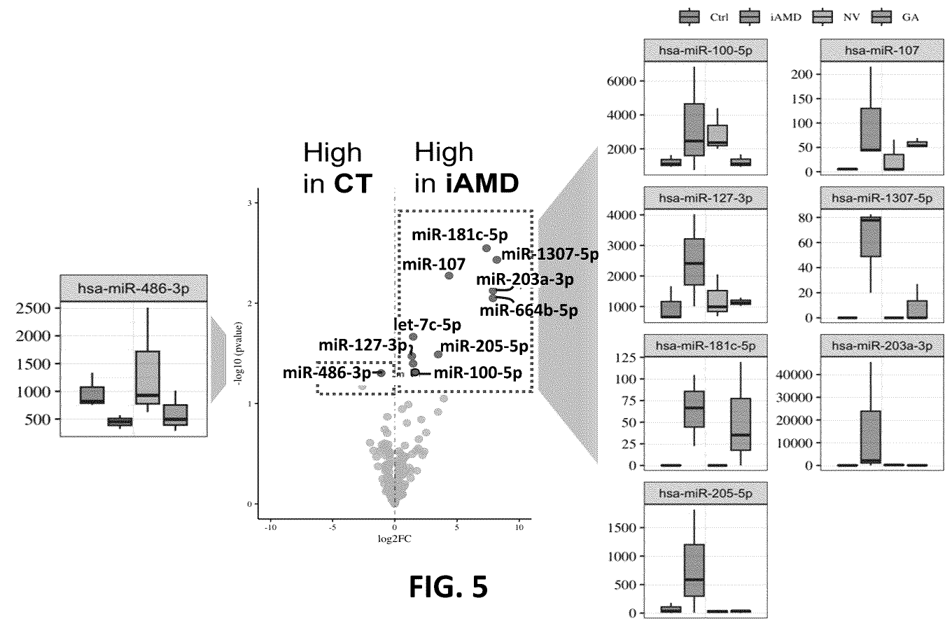

FIG. 5 includes a volcano plot (center) highlighting the miRNAs expressed highly in the control individuals versus iAMD patients. The diagram on the right shows the seven miRNAs expressed significantly higher in iAMD patients compared to healthy individuals (CT), namely: hsa-miR-100-5p, hsa-niR-107, hsa-niR-127-3p, hsa-niR-1307-5p, hsa-miR-181c-5p, hsa-miR-203a-3p, and hsa-miR-205-5p. The diagram on the left shows the sole miRNA expressed significantly higher in healthy individuals versus iAMD patients, hsa-miR-486-3p.

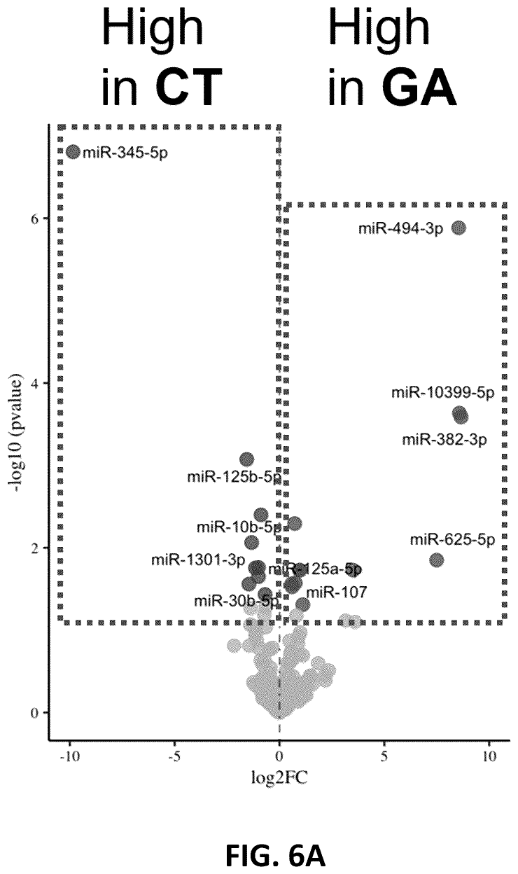

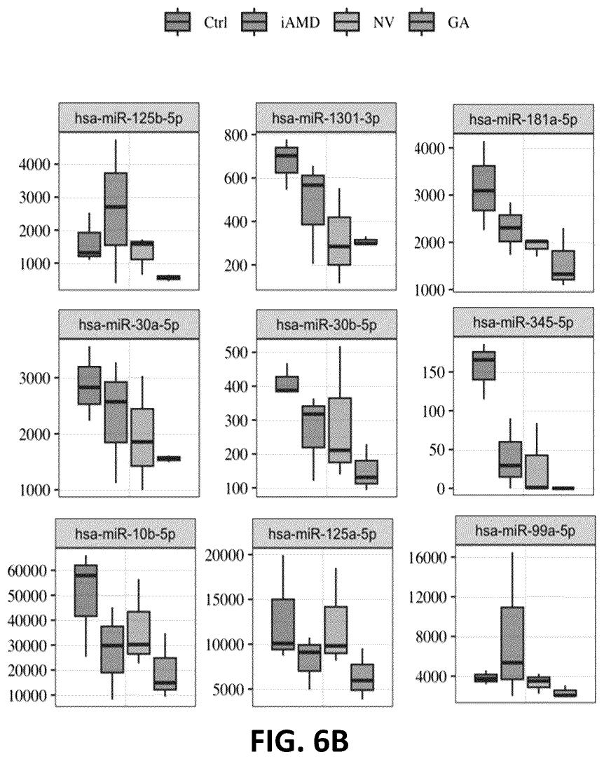

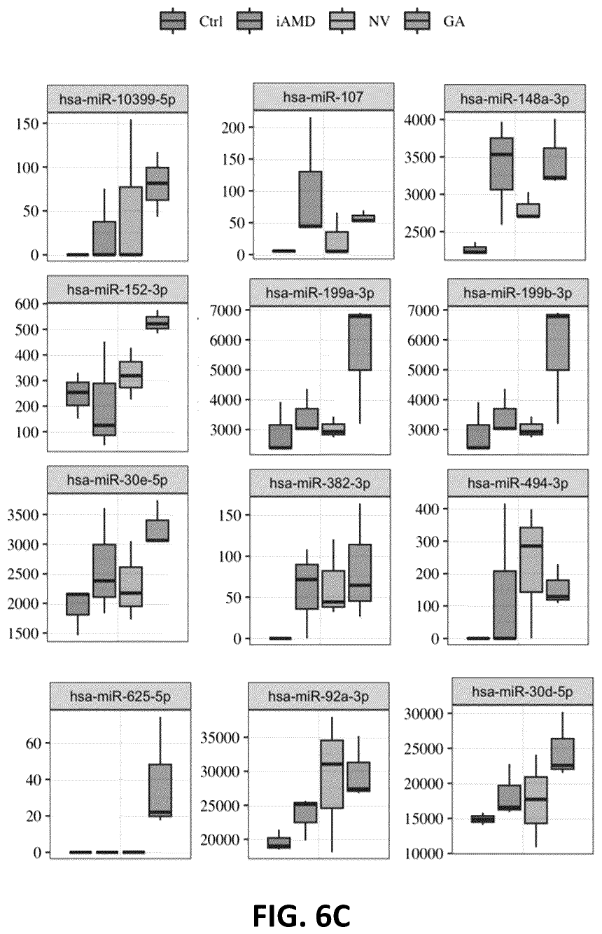

FIGS. 6A-6C depict miRNA expression in healthy individuals versus GA patients. In particular, FIG. 6A is a volcano plot highlighting the miRNAs expressed highly in the control individuals versus the GA patients. The diagram of FIG. 6B shows the nine miRNAs expressed significantly higher in healthy individuals (CT) compared to GA patients, namely: hsa-miR-125b-5p, hsa-miR-1301-3p, hsa-miR-181a-5p, hsa-miR-30a-5p, hsa-miR-30b-5p, hsa-miR-345-5p, hsa-miR-10b-5p, hsa-niR-125a-5p, and hsa-miR-99a-5p. The diagram of FIG. 6C shows the 12 miRNAs expressed significantly higher in GA patients compared to healthy individuals, namely: hsa-miR-10399-5p, hsa-miR-107, hsa-miR-148a-3p, hsa-miR-152-3p, hsa-miR-199a-3p, hsa-miR-199b-3p, hsa-miR-30e-5p, hsa-miR-382-3p, hsa-miR-494-3p, hsa-miR-625-5p, hsa-miR-92a-3p, and hsa-miR-30d-5p.

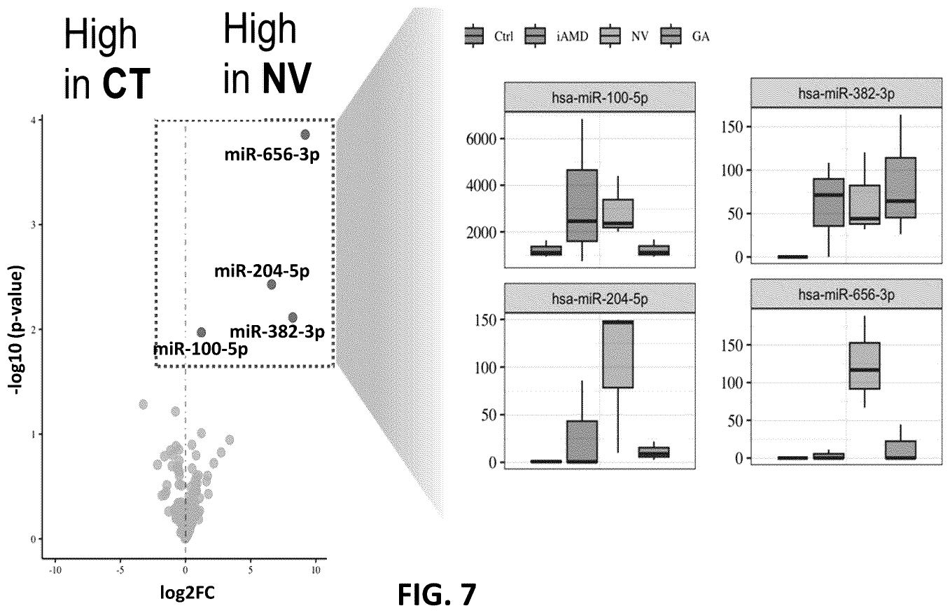

FIG. 7 includes a volcano plot highlighting the miRNAs expressed highly in the control individuals versus NV patients. As shown in the diagram on the right, four miRNAs were expressed significantly higher in NV patients compared to healthy individuals (CT), namely: hsa-miR-100-5p, hsa-miR-382-3p, hsa-miR-204-5p, and hsa-miR-656-3p.

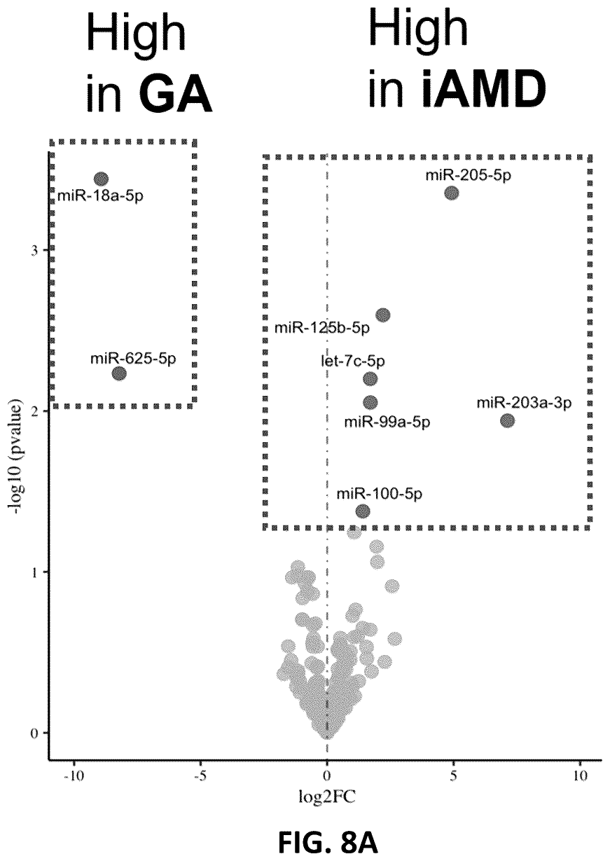

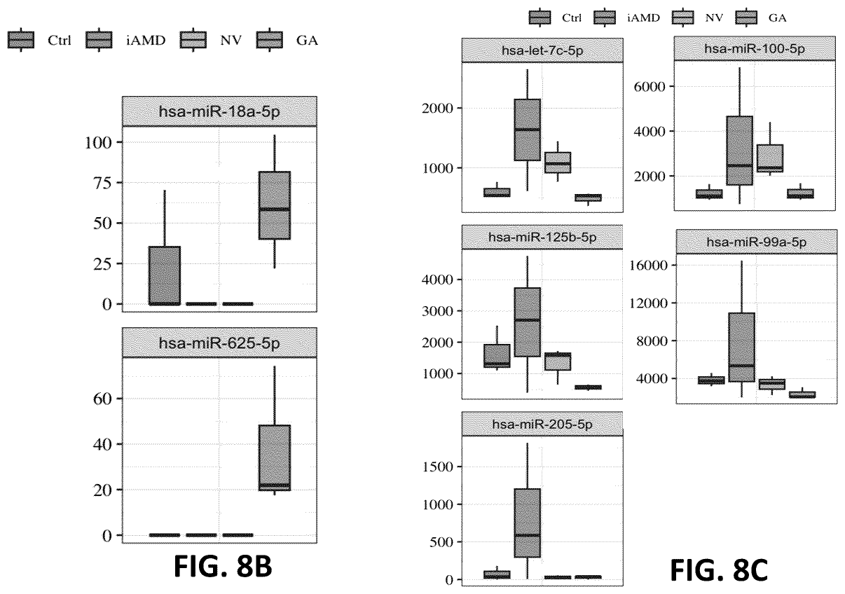

FIGS. 8A-SC depict miRNA expression in GA patients versus iAMD patients. In particular, FIG. 8A is a volcano plot highlighting the miRNAs expressed highly in GA patients versus iAMD patients. The diagram of FIG. 8B shows the two miRNAs expressed significantly higher in GA patients versus iAMD patients, namely: hsa-miR-18a-5p and hsa-miR-625-5p. The diagram of FIG. 8C shows the five miRNAs expressed significantly higher in iAMD patients compared to GA patients, namely: hsa-let-7c-5p, hsa-miR-100-5p, hsa-miR-125b-5p, hsa-11UR-99a-5p, and hsa-miR-205-5p.

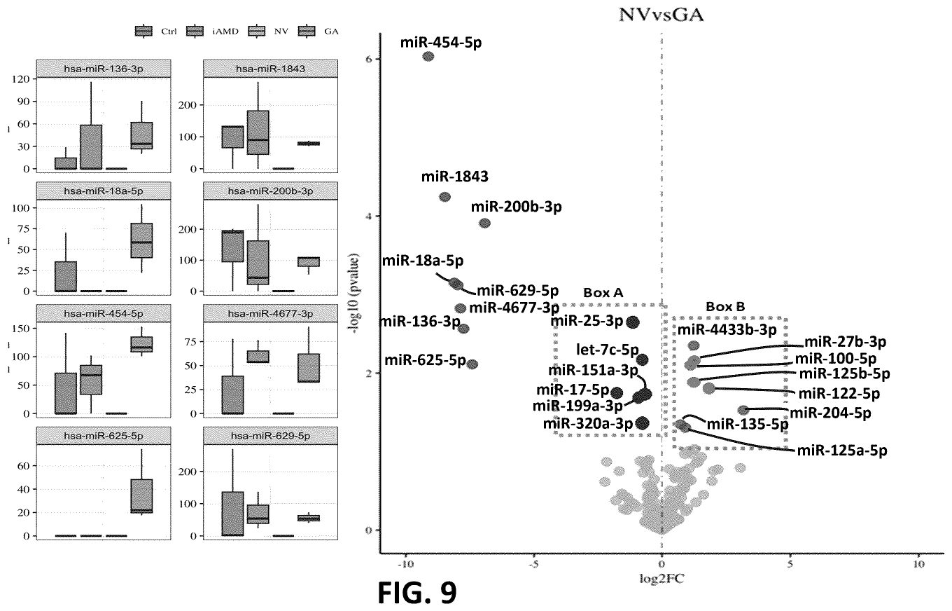

FIG. 9 includes a volcano plot highlighting the miRNAs expressed highly in the GA patients versus the NV patients. The diagram on the left shows the eight miRNAs expressed significantly higher in GA patients versus NV patients, namely: hsa-miR-136-3p, hsa-miR-1843, hsa-miR-18a-5p, hsa-miR-200b-3p, hsa-miR-454-5p, hsa-miR-4677-3p, hsa-miR-625-5p, and hsa-miR-629-5p. An additional six miRNAs, within box A were also expressed significantly higher in GA patients versus NV patients. A total of eight miRNAs, within box B, were expressed significantly higher in NV patients versus GA patients.

Tables 1A, 1B, and 1C, below, detail the results of metabolomics characterization of plasma-derived EVs collected from subjects afflicted with AMD. Among the 125 energy metabolites measured in the isolated EVs, ten exhibited differential abundance in AMD, including iAMD, NV, and GA, patients, relative to healthy controls. The ten metabolites included L-histidine, L-cystine, phosphoenolypyruvate, 2-oxoglutaramate, dimethylglycine tetrahydrofolate, L-citrulline, indolepyruvate, serotonin, and octenoyl-l-carnitine (acyl-C8:1). The median (Table 1A), fold change of AMD conditions versus the control (Table 1Bf), and P value for the significant proteins when comparing to control or healthy individuals (Table 1C) are provided.

TABLE 1A

| | Median | | | |
|---|---|---|---|---|
| Metabolite | Control | GA | iAMD | NV |
| L-histidine | 4.86E+05 | 5.09E+05 | 5.17E+05 | 7.42E+05 |
| L-cystine | 1.14E+05 | 1.11E+05 | 8.35E+04 | 1.36E+05 |
| phosphoenolypyruvate | 2.36E+04 | 2.89E+04 | 2.33E+04 | 2.93E+04 |
| 2-oxoglutaramate | 1.98E+04 | 4.40E+04 | 2.33E+04 | 4.19E+04 |
| dimethylglycine | 4.48E+05 | 4.51E+05 | 3.83E+05 | 8.24E+05 |
| tetrahydrofolate | 4.96E+03 | 7.79E+03 | 1.51E+04 | 1.20E+04 |
| L-citrulline | 2.70E+05 | 2.58E+05 | 4.46E+05 | 4.72E+05 |
| indolepyruvate | 4.68E+04 | 7.10E+04 | 5.71E+04 | 1.01E+05 |
| serotonin | 1.48E+05 | 1.63E+05 | 2.50E+05 | 2.17E+05 |
| octenoyl-l-carnitine | 1.27E+05 | 3.76E+05 | 1.32E+05 | 4.98E+05 |
| (acyl-C8:1) | | | | |

TABLE 1B

| | Fold Change/Control | | |
|---|---|---|---|
| Metabolite | GA | iAMD | NV |
| L-histidine | 1.05 | 1.06 | 1.52 |
| L-cystine | 0.97 | 0.73 | 1.19 |
| phosphoenolypyruvate | 1.22 | 0.99 | 1.24 |
| 2-oxoglutaramate | 2.23 | 1.18 | 2.12 |
| dimethylglycine | 1.01 | 0.85 | 1.84 |
| tetrahydrofolate | 1.57 | 3.04 | 2.42 |
| L-citrulline | 0.95 | 1.65 | 1.75 |
| indolepyruvate | 1.52 | 1.22 | 2.17 |
| serotonin | 1.10 | 1.70 | 1.47 |
| octenoyl-l-carnitine | 2.95 | 1.04 | 3.91 |
| (acyl-C8:1) | | | |

TABLE 1C

P value vs. Control

| Metabolite | GA | iAMD | NV |
|---|---|---|---|
| L-histidine | 8.49E−01 | 9.97E−01 | 3.60E−02 |
| L-cystine | 2.70E−01 | 3.53E−02 | 7.69E−01 |
| phosphoenolypyruvate | 8.98E−03 | 9.55E−01 | 2.26E−02 |
| 2-oxoglutaramate | 1.88E−01 | 6.56E−01 | 3.48E−02 |
| dimethylglycine | 4.99E−01 | 6.00E−01 | 4.59E−02 |
| tetrahydrofolate | 5.23E−01 | 1.82E−01 | 1.40E−02 |
| L-citrulline | 4.03E−01 | 2.23E−01 | 3.55E−02 |
| indolepyruvate | 2.75E−01 | 2.50E−01 | 4.94E−04 |
| serotonin | 7.79E−01 | 2.99E−01 | 3.95E−02 |
| octenoyl-l-carnitine | 2.33E−01 | 5.82E−01 | 8.42E−03 |
| (acyl-C8:1) | | | |

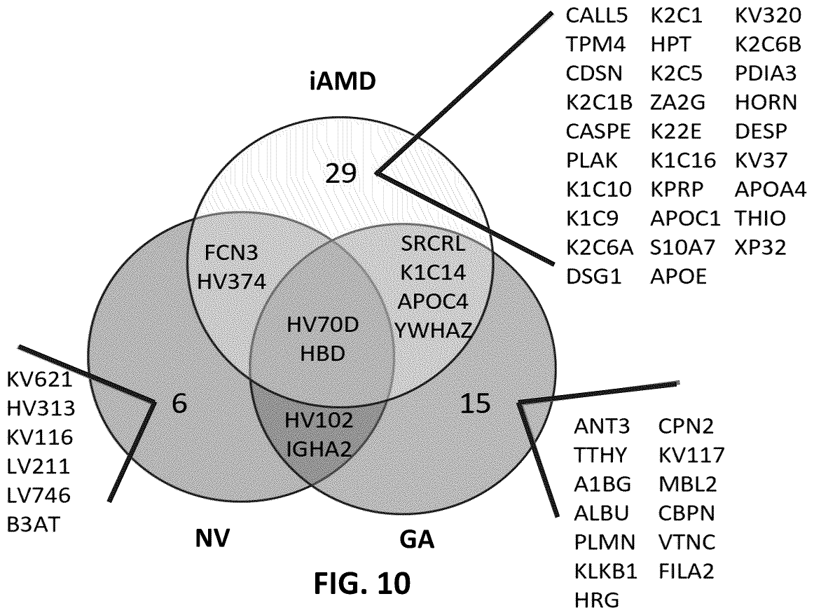

FIG. 10 is a Venn diagram showing the separate protein cargos obtained from within plasma-derived EVs collected from patients with iAMD, NV, and GA. As shown, 29 proteins were only expressed in iAMD patients, 15 proteins were expressed only in GA patients, and six proteins were expressed only in NV patients, with the expression of two proteins (HV70D) common to all AMD groups. The NV-only proteins were: KV621, HV313, KV116, LV211, LV746, and B3AT. The GA-only proteins were: ANT3, TTHY, A1BG, ALBU, PLMN, KLKB1, HRG, CPN2, KV1 17, MBL2, CBPN, VTNC, and FILA2. The iAMD-only proteins were: CALL5, TPM4, CDSN, K2CIB, CASPE, PLAK, K1C10, K1C9, K2C6A, DSG1, K2C1, HPT, K2C5, ZA2G, K22E, K1C16, KPRP, APOC1, S10A7, APOE, KV320, K2C6B, PDIA3, HORN, DESP, KV37, APOA4, THIO, and XP32.

By implementing the experimental methods described above, the top significant biomarkers of iAMD, AMD, NV, and GA were identified, including protein biomarkers, miRNA biomarkers, and metabolite biomarkers that were significantly different (p<0.05) when comparing healthy control individuals to individuals afflicted with iAMD, all age-related AMD, NV, and GA. Using plasma as the biofluid for analysis for each form of AMD (including all age-related AMD), mass spectrometry was used to identify protein biomarkers, RNA sequencing was used to identify the miRNA biomarkers, and metabolomic analysis was used to identify the metabolite biomarkers. In sum, biomarkers indicative of iAMD included CALL5, TPM4, CDSN, K2C1B, SSC5D, IMP4, FCN3, miR-181c-5p, miR-1307-5p, miR-203a-3p, and L-cystine. Biomarkers indicative of AMD included miR-107, miR-181a-5p, miR-148a-3p, and miR-769-5p. Biomarkers indicative of NV included SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and 2-oxoglutaramate. Biomarkers indicative of GA included SSC5D, YWHAZ, APOC4, ANT3, TTR, VTN, miR-494-3p, miR-10399-5p, miR-625-5p, and phosphoenolpyruvate.

Area under the curve (AUC) was determined for a subset of biomarkers to determine the sensitivity and specificity of each marker. In particular, the AUC values for iAMD markers CALL5, FCN3, TPM4, CDSN and K2C1B were 0.97 (sensitivity=100; specificity=75), 0.63 (sensitivity=100; specificity=35), 0.86 (sensitivity=85; specificity=100), 0.88 (sensitivity=88; specificity=100), and 0.93 (sensitivity=85; specificity=100), respectively. The AUC values for GA biomarkers VTNC, SRCRL, TTHY were 0.73 (sensitivity=80; specificity=80), 0.96 (sensitivity=90; specificity=100), and 1.00 (sensitivity=100; specificity=100), respectively.

While multiple embodiments are disclosed herein, still other embodiments of the present disclosure will become apparent to those skilled in the art from the foregoing description. As will be apparent, the disclosed embodiments are capable of modifications in various obvious aspects, all without departing from the spirit and scope of this disclosure. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

1. A method of age-related macular degeneration assessment, comprising:
obtaining a biological sample from a subject;
capturing at least one biomarker indicative of one or more subtypes of age-related macular degeneration from the biological sample;
determining a presence or a level of the at least one biomarker in the biological sample; and
comparing the presence or the level of the at least one biomarker to a reference presence or level to determine whether the subject has age-related macular degeneration.

2. The method of claim 1, wherein the age-related macular degeneration comprises intermediate age-related macular degeneration.

3. The method of claim 2, wherein the at least one biomarker comprises one or more of CALL5, TPM4, CDSN, K2C1B, SSC5D, IMP4, FCN3, miR-181c-5p, miR-1307-5p, miR-203a-3p, and L-cystine.

4. The method of claim 1, wherein the age-related macular degeneration comprises neovascular age-related macular degeneration.

5. The method of claim 4, wherein the at least one biomarker comprises one or more of SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and 2-oxoglutaramate.

6. The method of claim 1, wherein the age-related macular degeneration comprises geographic atrophy.

7. The method of claim 6, wherein the at least one biomarker comprises one or more of SSC5D, YWHAZ, APOC4, ANT3, TTR, VTN, miR-494-3p, miR-10399-5p, miR-625-5p, and phosphoenolpyruvate.

8. The method of claim 1, wherein the at least one biomarker comprises one or more of miR-107, miR-181a-5p, miR-148a-3p, and miR-769-5p.

9. The method of claim 1, wherein the biological sample comprises a biofluid.

10. The method of claim 9, wherein the biofluid comprises plasma.

11. The method of claim 1, wherein capturing the at least one biomarker from the biological sample comprises isolating extracellular vesicles present within the biological sample.

12. The method of claim 11, further comprising identifying a cargo contained within the extracellular vesicles, the cargo comprising protein, miRNA, and metabolites.

13. The method of claim 1, wherein obtaining the biological sample and measuring the level of the at least one biomarker occurs at point of care.

14. The method of claim 1, further comprising determining whether administering a drug formulated to treat age-related macular degeneration will be effective.

15. The method of claim 1, further comprising implementing a treatment based on whether the subject is afflicted with a particular subtype of age-related macular degeneration.

16. A system for identifying a subject afflicted with age-related macular degeneration, comprising:
a device for receiving a biological sample from a subject suspected of having one or more subtypes of age-related macular degeneration;
a device for identifying the presence of least one biomarker in the biological sample obtained from the subject; and
a device for measuring the level of the at least one biomarker in the biological sample.

17. The system of claim 16, wherein the biological sample is a fluid sample.

18. The system of claim 17, wherein the fluid sample comprises plasma, urine, tears, saliva, aqueous humor, vitreous, or serum.

19. The system of claim 16, wherein the one or more subtypes of age-related macular degeneration comprise intermediate age-related macular degeneration, neovascular age-related macular degeneration, and geographic atrophy.

20. The system of claim 16, wherein the at least one biomarker comprises one or more of CALL5, TPM4, CDSN, K2C1B, SSC5D, IMP4, FCN3, miR-181c-5p, miR-1307-5p, miR-203a-3p, and L-cystine, SLC4A1, LV746, KV621, miR-656-3p, miR-204-5p, miR-382-3p, indol pyruvate, octenoyl-l-carnitine, tetrahydrofolate, phosphoenolpyruvate, eicosapentaenoic acid, dimethylglycine, serotonin, L-citrulline, L-histidine, and 2-oxoglutaramate, YWHAZ, APOC4, ANT3, TTR, VTN, miR-494-3p, miR-10399-5p, and miR-625-5p.
